(19) United States
(12) Patent Application Publication
(10) Pub. No.: US 2024/0407923 A1
(43) Pub. Date: Dec. 12, 2024

(54) Mandible Graft Reconstruction Assembly, System and Procedure

(71) Applicants: Baber Khatib, Portland, OR (US); Justin Lyons, Jacksonville, FL (US); Andrew Strickland, Jacksonville, FL (US); Scott Eibel, Jacksonville, FL (US); Matt Radloff, Jacksonville, FL (US)

(72) Inventors: Baber Khatib, Portland, OR (US); Justin Lyons, Jacksonville, FL (US); Andrew Strickland, Jacksonville, FL (US); Scott Eibel, Jacksonville, FL (US); Matt Radloff, Jacksonville, FL (US)

(21) Appl. No.: 18/736,073

(22) Filed: Jun. 6, 2024

Related U.S. Application Data
(60) Provisional application No. 63/471,475, filed on Jun. 6, 2023.

(51) Int. Cl. A61F 2/28; A61B 34/10; A61C 8/00; A61F 2/30

(52) U.S. Cl. CPC A61F 2/2803; A61B 34/10; A61C 8/0022; A61C 8/0089; A61F 2/3094; A61B 2034/105; A61B 2034/107; A61B 2034/108

(57) ABSTRACT

A method for designing and producing a reconstruction assembly for implantation onto a mandible to replace a resected or damaged portion, the method utilizing a virtual and a physical 3-D model of a patient, as well as the reconstruction assembly itself, the reconstruction assembly having a fixation implant, a fibular flap, implant posts and a dental prosthesis with removable positioning extensions.

### BACKGROUND OF THE INVENTION

This invention relates generally to facial reconstruction implant systems and procedures wherein 3-D virtual and/or physical models are used, such as for example in bone reconstruction, orthognathic surgery or related bone treatments involving osteotomies or bone repair, and in particular to jaw replacement surgery using patient-specific models, i.e., models customized and configured based on 3-D computer-aided design to recreate in model form a precise 3-D copy of the maxilla or mandible of the patient. The invention also relates to the method of designing, manufacturing and utilizing such models in a surgical procedure wherein a portion of the maxilla or mandible is replaced by an assembly comprising a dental prosthesis, a fibula flap, implant posts, and a fixation implant, the model ensuring that the assembly is properly configured and oriented prior to implantation in the patient. The invention also relates to the assembly itself, wherein the fixation implant is adapted to be fastened to the mandible, the dental prosthesis is adapted to be fastened to the mandible and also connected to the fibula flap by the implant posts, and wherein the fibula flap is fastened to the fixation bone plate.

A mandible may be malformed, diseased, fractured or otherwise damaged to the extent that it needs to be surgically repaired by removing a portion of the bone, orienting segments of fibula bone to recreate a reconstruction arch or flap, securing a dental prosthesis to the reconstruction flap, and securing the combined reconstruction flap and dental prosthesis to the patient's remaining mandible segments. Modern surgery makes use of computer-aided design and manufacturing techniques whereby surgeons and technicians create virtual 3-D models of a patient's bone structure in order to properly design, size, orient and configure the reconstruction assembly. A representative procedure is discussed in detail in an article titled “Fibula Jaw in a Day: State of the Art in Maxillofacial Reconstruction” (J Oral Maxillfac Surg 74:1284.e1-1284.e15, 2016), the disclosure of which is incorporated herein by reference.

It is very important that the reconstructed mandible reconstruction assembly be properly positioned and oriented relative to the patient's mandible upon affixation in order to preserve the patient's previous or desired jaw articulation and junction between the opposing dental arches. It is an object of this invention to provide a novel system, procedure and reconstruction assembly that accomplishes this goal.

### BRIEF DESCRIPTION OF THE DRAWINGS

FIG. 1 illustrates a 3-D physical model of a resected mandible provided with positioning recesses to receive the dental prosthesis and screw apertures for affixation of the dental prosthesis.

FIGS. 2 and 3 illustrate a dental prosthesis with anterior and posterior positioning extensions, the positioning extensions being seated in the mandible positioning recesses.

FIG. 4 illustrates the dental prosthesis of FIGS. 2 and 3 mounted on an articulating 3-D physical model showing proper alignment relative to the upper teeth.

FIG. 5 illustrates a reconstruction assembly mounted on the mandible of a 3-D physical articulating model, the reconstruction assembly comprising a dental prosthesis having anterior and posterior positioning extensions positioned in the mandible positioning recesses, a fixation implant affixed to the mandible, a fibula flap affixed to the fixation implant with implant posts extending from the fibula flap into the bores of the dental prosthesis.

### SUMMARY OF THE INVENTION

The invention is in general a process or method for modeling and producing a reconstruction assembly for implantation onto a mandible to replace a resected or damaged portion, as well as the reconstruction assembly itself. The use of 3-D modeling and implant design and development using computer assisted design is well known in the art. A representative reconstruction procedure utilizing 3-D modelling is discussed in detail in an article titled “Fibula Jaw in a Day: State of the Art in Maxillofacial Reconstruction” (J Oral Maxillfac Surg 74:1284.e1-1284.e15, 2016), the disclosure of which is incorporated herein by reference.

The method comprises:
(a) creating a virtual 3-D model of a patient's mandible that has been resected,
(b) designing components of a reconstruction assembly comprising a fixation implant contoured to the surface configuration of the patient's remaining bone, a dental prosthesis, anterior and posterior positioning extensions joined to the dental prosthesis, implant posts, and a fibula flap,
(c) determining the location for positioning recesses to receive the anterior and posterior positioning extensions and screw apertures for affixation of the fixation implant in the virtual 3-D model,
(d) creating a physical 3-D model of a patient's mandible that has been resected, the physical 3-D model including the positioning recesses to receive the anterior and posterior positioning extensions and screw apertures for affixation of the fixation implant
(e) manufacturing the fixation implant, dental prosthesis, anterior and posterior positioning extensions joined to the dental prosthesis, and implant posts,
(f) inserting the implant posts into a fibula,
(g) harvesting segments from the fibula,
(h) affixing the fibula segments to the fixation implant to create a fibula flap,
(i) positioning the dental prosthesis onto the physical model such that the positioning extensions are received by the positioning recesses,
(j) affixing the fixation implant and fibula flap onto the 3-D model with the implant posts extending into bores in the dental prosthesis,
(k) applying resin to cement the dental prosthesis to the implant posts, the positioning extensions ensuring the correct location and orientation of the dental prosthesis relative to the fixation implant,
(l) removing the positioning extensions from the dental prosthesis, and
(m) transitioning the reconstruction assembly from the 3-D physical model onto the patient by securing the fixation implant onto the patient's bone.

The anterior and posterior positioning extensions and dental prosthesis are preferably manufactured as a single unit, with the positioning extensions comprising one or more arms extending from the dental prosthesis, the arms connected to seating members designed to fit within the positioning recesses on the 3-D physical model.

### DETAILED DESCRIPTION OF THE INVENTION

The invention in various embodiments is a process or method for modeling and producing a reconstruction assembly for implantation onto a mandible to replace a resected or damaged portion, as well as the reconstruction assembly itself and the method of replacing a resected or damaged portion of a mandible.

Using known 3-D imaging or scanning technology and methodology, a virtual 3-D model of the actual patient's jaw region is created having a portion of the mandible removed. A virtual reconstruction assembly is then designed based on the virtual 3-D model, the virtual reconstruction assembly comprising a fixation implant contoured to the surface configuration of the patient's remaining bone, a dental prosthesis, anterior and posterior positioning extensions joined to the dental prosthesis, and implant posts. The location of positioning recesses to receive the anterior and posterior positioning extensions of the dental prosthesis and the location for screw apertures for affixation of the fixation implant are determined in the virtual 3-D model.

A physical 3-D model 90 of a patient's jaw region matching the virtual 3-D model is created, the physical 3-D model 90 including the positioning recesses 91 to receive the anterior and posterior positioning extensions 60 of the dental prosthesis 20 and the screw apertures 31 for affixation of the fixation implant. The actual fixation implant 30, dental prosthesis 20, anterior and posterior positioning extensions 60 joined to the dental prosthesis 30, and implant posts 50 are then manufactured.

The fibula flap 40 is created in known manner by inserting the implant posts 50 into the fibula bone prior to cutting the bone into segments, harvesting the segment or segments, and affixing the fibula segments to the fixation implant 30 to create the fibula flap 40.

The dental prosthesis 20 is placed onto the physical 3-D model 90 such that the positioning extensions 60 are received within the positioning recesses 91, thereby properly positioning the dental prosthesis 20 relative to the upper jaw and teeth. The fixation implant 30 and fibula flap 40 are temporarily affixed onto the 3-D model by inserting screws through the apertures 31 into the model apertures 92, with the implant posts 50 positioned to extend into the bores 23 in the dental prosthesis 20. In this manner, the dental prosthesis will be properly oriented relative to the upper jaw. Resin is then applied into the bores 23 to cement the dental prosthesis 20 to the implant posts 50.

The positioning extensions 60 are removed from the dental prosthesis 20 and the reconstruction assembly 10 is then transitioned onto the mandible of the patient by securing the fixation implant 30 onto the patient's bone using screws.

FIG. 1 illustrates a 3-D model 90 of the patient's resected mandible. In this example, a patient has had a section of the mandible body between the ramus and the symphysis (chin) resected, and the 3-D model 90 is created to match the configuration of the resected mandible. The 3-D model 90 is provided with one or more anterior and one or more posterior positioning recesses 91, in this illustration a pair of each, as well as apertures 92 for alignment of the fixation implant 30.

The reconstruction assembly 10 is designed and manufactured using CAD or similar computer programs. The reconstruction assembly 10 comprises a dental prosthesis 20, a fixation implant 30, a fibula flap 40 and implant posts 50.

The dental prosthesis 20 is an artificial member provided with teeth 21 and tissue 22 to replace the resected elements. It is important to properly locate the dental prosthesis 20 on the resected mandible, and therefore the dental prosthesis 20 is provided with removable anterior and posterior positioning extensions 60, with the dental prosthesis 20 and the positioning extensions 60 most preferably manufactured as a single unit. The positioning extensions 60 comprise one or more connector arms 61 each extending from the dental prosthesis tissue 22 to a seating member 62. The size, configuration and location of the seating members 62 match the positioning recesses 91 of the mandible model 90. The dental prosthesis 20 is also provided with bores 23 to receive the implant posts 50 extending from the fibula flap 40.

The fixation implant 30 is a rigid plate, frame or similar construct that conforms to the surface configuration of the patient's non-resected mandible bone and is provided with apertures 31 to receive bone screws or similar mechanical fixation members. The purpose of the fixation implant 30 is to secure the reconstruction assembly 10 to the patient.

In known manner, a fibula cutting guide is mounted onto a fibula and the implant posts 50 are inserted into the fibula. The guide ensures that the implant posts 50 are correctly located and aligned in the fibula flap 60 once the fibula is osteotomized. The one or more fibula segments are obtained and mounted to the fixation implant 30 in the proper location using bone screws or the like, thereby creating a fibula flap 60 onto which the dental prosthesis 20 will be mounted.

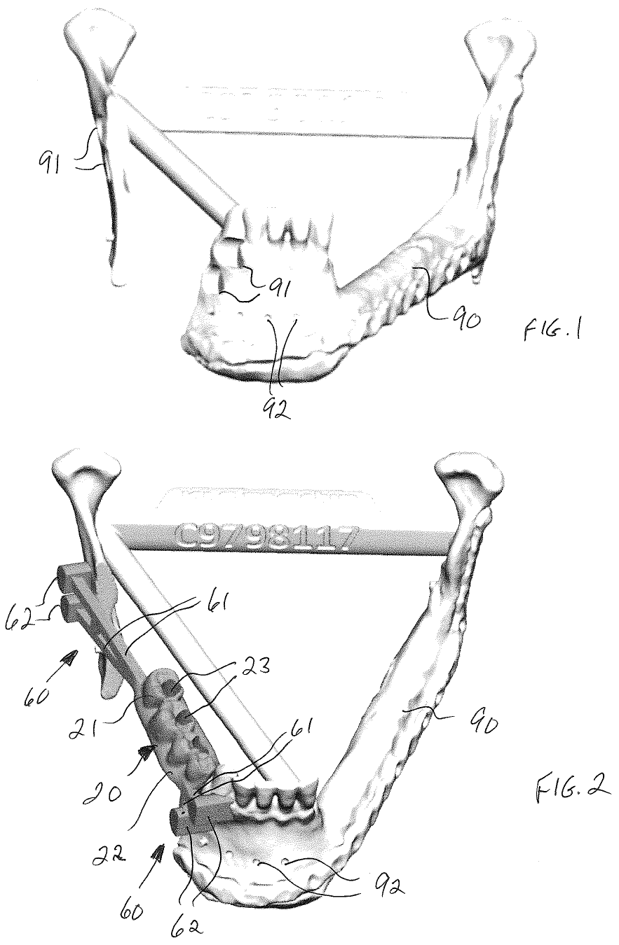

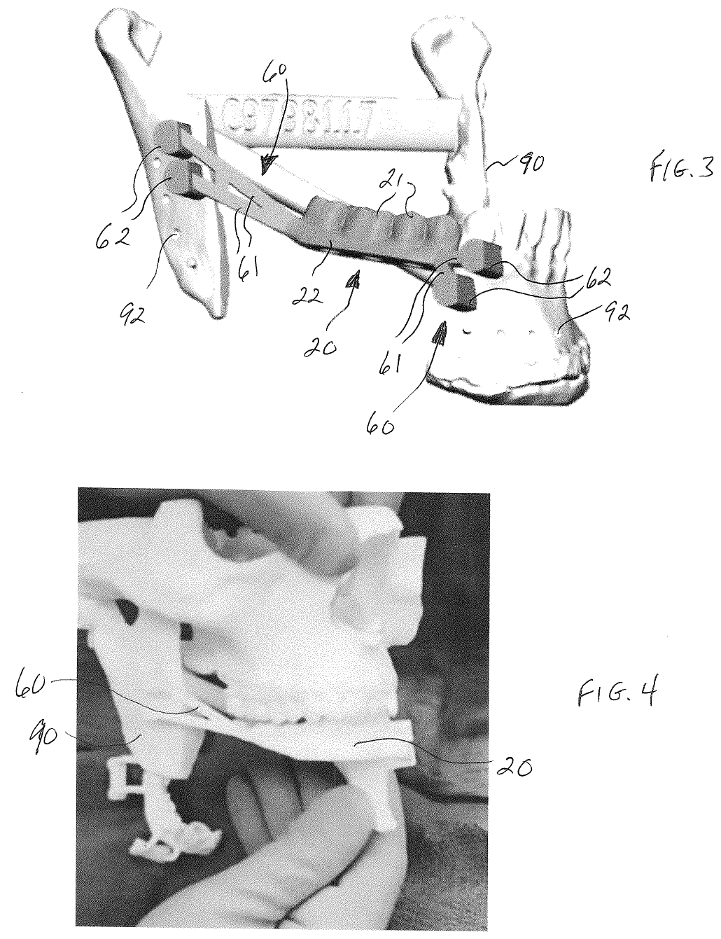

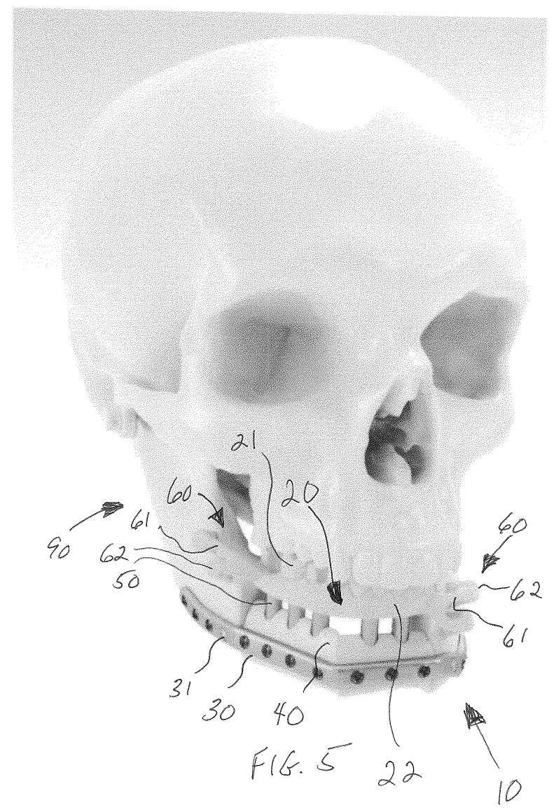

The fixation implant 30 is temporarily affixed to the 3-D model by inserting screws into the model apertures 92. The dental prosthesis 20 is properly positioned on the 3-D model by placing the seating members 62 in the model positioning recesses 91, as shown in FIGS. 2 and 3, with the implant posts 50 residing in the dental prosthesis bores 23. Resin or other cementing material is applied into the bores 23 to secure the dental prosthesis 20 to the implant posts 50 and thereby to the fibula flap 60.

Because the components have been precisely designed, the positioning extensions 60 ensure that the dental prosthesis 20 is properly located relative to the fixation implant 30 as well as to the teeth and upper jaw of the model 90. Once the resin has cured, the positioning extensions 60 are removed from the dental prosthesis 20 by cutting or similar means. This leaves the finished reconstruction assembly 10, comprised of the fixation implant 30, the fibular flap 60, the implant posts 50 and the dental prosthesis 20.

The reconstruction assembly 10 is then removed from the 3-D model 90 and transferred to the patient. The fixation implant 30 is now permanently affixed to the patient's bone using bone screws or the like, thereby correctly locating the dental prosthesis 20.

It is understood that substitutions or equivalents to some steps or elements set forth above may be obvious to those of skill in the art, and therefore the true scope and definition of the invention is to be as set forth in the following claims.

We claim:

1. A mandible reconstruction method comprising the steps of:
(a) creating a virtual 3-D model of a patient's mandible that has been resected,
(b) designing components of a reconstruction assembly comprising a fixation implant contoured to the surface configuration of the patient's remaining bone, a dental prosthesis, anterior and posterior positioning extensions joined to the dental prosthesis, implant posts, and a fibula flap,
(c) determining the location for positioning recesses to receive the anterior and posterior positioning extensions and screw apertures for affixation of the fixation implant in the virtual 3-D model,
(d) creating a physical 3-D model of a patient's mandible that has been resected, the physical 3-D model including the positioning recesses to receive the anterior and posterior positioning extensions and screw apertures for affixation of the fixation implant
(e) manufacturing the fixation implant, dental prosthesis, anterior and posterior positioning extensions joined to the dental prosthesis, and implant posts,
(f) inserting the implant posts into a fibula,
(g) harvesting segments from the fibula,
(h) affixing the fibula segments to the fixation implant to create a fibula flap,
(i) positioning the dental prosthesis onto the physical model such that the positioning extensions are received by the positioning recesses,
(j) affixing the fixation implant and fibula flap onto the 3-D model with the implant posts extending into bores in the dental prosthesis,
(k) applying resin to cement the dental prosthesis to the implant posts, the positioning extensions ensuring the correct location and orientation of the dental prosthesis relative to the fixation implant,
(l) removing the positioning extensions from the dental prosthesis, and
(m) transitioning the reconstruction assembly from the 3-D physical model onto the patient by securing the fixation implant onto the patient's bone.

2. A mandible reconstruction assembly comprising:
(a) a fixation implant contoured to the surface configuration of a patient's bone and adapted to be fastened thereon,
(b) a dental prosthesis having bores,
(c) one or more removable positioning extensions temporarily joined to the dental prosthesis, the one or more removable positioning extensions adapted to be received within positioning recesses formed on a 3-D physical model of the patient's mandible,
(d) a fibula flap affixed to the fixation implant, and
(e) implant posts affixed to the fibula flap and configured to reside within the bores of the dental prosthesis, the implant posts being cemented to the dental prosthesis.
